(19) United States
(12) Patent Application Publication
(10) Pub. No.: US 2004/0059196 A1
(43) Pub. Date: Mar. 25, 2004

(54) Patient monitoring system for the automatic registration of restrictions on daily abilities

(75) Inventors: Klaus Abraham-Fuchs, Erlangen (DE); Johannes Bieger, Munchen (DE); Arne Hengerer, Erlangen (DE); Eva Rumpel, Erlangen (DE); Daniel Tietze, Spardorf (DE)

Correspondence Address: YOUNG & THOMPSON, 745 SOUTH 23RD STREET 2ND FLOOR, ARLINGTON, VA, 22202

(73) Assignee: SIEMENS AKTIENGESELLSCHAFT, Munchen (DE)

(21) Appl. No.: 10/335,883

(22) Filed: Jan. 3, 2003

(30) Foreign Application Priority Data
Sep. 25, 2002 (EP) 02021476.3

(51) Int. Cl. A61B 5/00

(52) U.S. Cl. 600/300

(57) ABSTRACT

A patient monitoring system and method for the registration of restrictions of patient's abilities in daily activities with the aid of a list of questions, wherein an expert system which, via an electronic transmission path, automatically and regularly presents each patient with individual questions or subcombinations from the list of questions which take into account his personal conditions and/or his medical history and documents and evaluates the replies and, from this, if necessary, derives new specific questions to the patient.

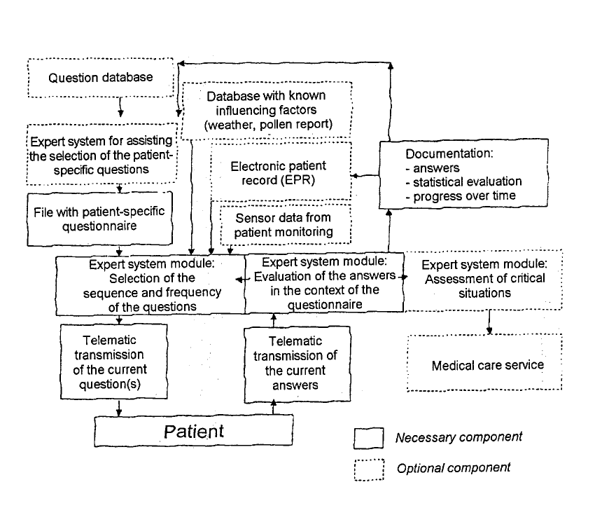

### FIELD OF THE INVENTION

The invention relates to a patient monitoring system for the registration of restrictions on daily abilities with the aid of a list of questions comprising a large number of questions.

### BACKGROUND OF THE INVENTION

Patients are increasingly being looked after with the aid of telemedical forms of care, for example during the management of chronic illnesses (for example diabetes, asthma, Alzheimer's, depression), in aftercare following stationary rehabilitation (for example stroke) or in order to accompany persons requiring care. In order to support the decision-making of the specialist medical personnel providing the care, medically relevant data (for example blood pressure, blood sugar values, ECS) are registered on the patient at home and transmitted to the physician by remote data transmission.

In order to assess how well the patient is managing with his illness in daily life at home, however, not only are physiological measured values important but also knowledge about the quality with which the patient can carry out important and typical routines in daily life. Such questions are, for example:

Can you climb the stairs to the first floor without hardship?

Did you have problems getting dressed today?

Can you manage with the shower?

Have you had problems with buying necessary food?

Questionnaires, which register the quality with which the patients can carry out these typical “activities of daily living” are already available in various embodiments as a standard and, typically, comprise about 15 to 50 questions. They are generally designated questionnaires for the “registration of the quality of living”, and are typically used in the context of clinical studies. However, this is normally done only episodically at long time intervals, for example at the start and at the conclusion of a clinical study. In addition, during such a clinical study, it is not possible for all the questions required to register the quality of living to be posed, since overall hundreds of questions could be posed for this purpose, from which specific questionnaires always extract only a group of at most fifty questions, depending on the type of complaint. However, it would certainly be desirable, in order to assess the condition of a patient, if even if possibly only now and then—quite different questions about the state of health could be posed from the large overall list of questions.

However, in the event of telemedical care, it would also be advantageous if the development of these daily capabilities could be monitored continuously (“monitoring of the quality of living”), in particular with a view to the early detection of a deterioration. For this purpose, however, it is not possible to have the patient answer the entire questionnaire, daily or even only weekly, since the patient would not accept this amount of effort. In exactly the same way, it is impossible for reasons of cost to carry out the registration of this information interactively with medical care personnel.

Previously, attempts have been made to solve this problem by means of repeated visits to and assessment by the therapist or else on the basis of self-estimation of the patient, who was registered by means of a questionnaire relating to quality of living or by keeping a patient diary or—in infrequent cases—using complicated sensor systems. The self-estimation of the patient is hardly suitable for data acquisition, however, since the patients (in particular those with neurological or psychiatric problems) cannot assess themselves objectively. Experience shows that patient diaries are not kept reliably over an extended time period. Typical sensor solutions are complicated in technical terms and in use and are able to supply only a limited picture, since they normally only measure the restriction itself, that is to say the mobility of joints, the reaction times to specific events and so on, and leave any compensation mechanisms which may be present unconsidered. Consequently, only infrequently are reliable databases available for the assessment of the result of therapy. In most cases, after the patient has been discharged from the therapy, no data acquisition takes place at all, since the expenditure associated with this is too high in the case of the methods used currently.

DE 196 37 383 A1 has already disclosed a date acquisition and evaluation device in which the state of a person is registered by sensors and, via an evaluation unit for assessing the situation of this person as compared with their usual daily routine, is assessed by taking account of further data, such as the time of day and the place of residence. However, such a sensor registration and evaluation system just does riot take into account the restrictions on daily capabilities, which cannot be acquired by means of sensors, such as

Have you suffered pain?

Did you sleep poorly?

Do you feel weak?

Do you lack appetite?

Have you had a bowel motion, if appropriate have you had diarrhea?

Do you have difficulties in reading the newspaper?

However, it is precisely this restriction on daily capabilities which can provide the significant approaches to a therapy and, if appropriate, also a change to a therapy.

### SUMMARY OF THE INVENTION

The invention is therefore based on the object of providing a patient monitoring system for the automatic registration of the restrictions on abilities in daily activities with the aid of a list of questions comprising a large number of questions which operates simply and reliably and does not make excessive demands on the patient, so that there is no fear that he or she will refuse to cooperate,

In order to achieve this object, such a patient monitoring system is characterized by an expert system which, via electronic transmission paths such as telephone, SMS, e-mail or the like, automatically and regularly presents each patient to be looked after with individual questions or subcombinations from the list of questions which take into account his personal conditions and/or his medical history and documents and evaluates the replies and, from this, it necessary derives new specific questions to the patient.

The data acquired in this way are extremely helpful in order to assess the success of therapy and in order, if appropriate, specifically to initiate further-reaching therapeutic measures.

In this case, the invention is based on the finding that, from a medical point of view, it is not necessary to answer all the questions in a quality-of-living list of questions in one go in one session. Likewise, the time interval at which specific questions have to be posed again, because the situation of the patient could have changed, may possibly be very different. The invention is therefore based on the fact that obtaining medically practical information can also be achieved for example by only one or two questions being posed each day, or else only each second or third day, and the questions should not be posed with the same frequency and in a fixed sequence either. Instead, the sequence and frequency of the questions should be adapted and individualized to the history of the disease and the current situation, or derived from the answers to the questions posed last.

For this reason, it is expedient to configure the patient monitoring system in such a way that, for each patient, a list of questions which takes into account his or her personal conditions and disease history is drawn up, for which purpose it is in turn expedient for the expert system to have access to a central or decentral electronic patient record and also to the sensor data from a patient monitoring system.

Since other influencing factors can also have a great influence on the wellbeing and the daily capabilities of a patient, in a refinement of the invention, the expert system should be connected to databases relating to known influencing factors, such as weather, the pollen report or the like.

A patient monitoring system according to the invention may expediently be integrated into an automated call center.

### BRIEF DESCRIPTION OF THE DRAWING

Further advantages, features and details of the invention emerge from the following description of an exemplary embodiment and by using the drawing, which represents a flow chart of the patient monitoring system.

### DETAILED I)ESCRIPTION OF THE INVENTION

For each patient, an individualized list of questions in electronic form (for example file, database, database query) is compiled, either by means of new compilation or by selection from a suitable question database. In this case, this selection from a comprehensive question database is expediently carried out via an expert system which, by using the specific data about a patient made available to it, including the prehistory of the patient's illness, compiles a practical selection of a list of questions possibly comprising 10 to 50 or more questions, which contains virtually all the questions which are necessary and meaningful for the respective patient and the assessment of the restrictions on his or her daily capabilities.

Then, from the electronic, patient-specific list of questions, taking into account rules relating to the time sequence with which these questions are to be posed and, if appropriate, further information from any electronic patient record which may be present, a computer-aided expert system selects one or possibly even a number of questions which are to be posed to the patient on this day. These rules can be controlled statically (question k on the nth day of a question cycle) or dynamically (for example, if question x is answered with yes, pose question y on the following day, and then continue with the static list). The dynamic rules can also be controlled by means of data obtained additionally, for example blood pressure values acquired telemedically.

The selected question is transmitted to the patient by electronics, for example telephone, SMS, e-mail or the like, and the patient is requested to answer the question. The answer should preferably consist only of yes, no or don't know or at most a marking on a scale, for example 1 to 3 or 0 to 5. The answer is entered by patients via a telephone keypad or sent back in an e-mail reply. optionally, generally known influencing factors, such as the weather, the time of year, known epidemics, pollen report and so on, can be taken into account in selecting the questions, but also in evaluating the answers to the questions.

The receiving computer of the patient monitoring system accepts the answer and enters it into a patient database, from which, inter alia, a “quality of living diary” of the patient may be compiled. In addition, for this purpose an expert system can evaluate the answers and, in the event of situations which appear to be medically critical, can notify an associated medical care service.

Furthermore, in the case of a telephone call center, the patient can be given the possibility of being connected to an adviser instead of the call-center computer by pressing a specific key.

In addition to answering the question just posed, the patient also has the possibility of entering additional information on his or her own, for which purpose this additional information is preferably selected from predefined lists, so that it can likewise be processed automatically.

While the answers to individual questions, taken on their own, do not permit a fundamental picture of the daily capabilities, the adapted selection and the regular acquisition over a relatively long time period supplies a relatively meaningful analysis. In this case, a relatively high degree of objectivity is also achieved as a result of the fact that a large number of parameters are acquired indirectly, and in this way the patient cannot be manipulated consciously or unconsiously. If necessary, the questions can be combined with and supplemented by data acquisition by sensors, for example bending sensors, a computer test of cognitive capabilities, etc. For the purpose of comprehensive evaluation of all the data collected, the user, for example the therapist, the disease manager, the medical insurance provider or the like, can obtain information on the development of the daily capability of the patient by means of a statistical module.

Finally, continuous long-term monitoring of the quality of living of the patients in rehabilitation aftercare offers the possibility of measuring the success of therapy and therefore the quality of the rehabilitation performance quantitatively in an objective and statistically significant manner.

1. A method of monitoring restrictions of a patient's abilities, the method comprising the steps of:
compiling in a database plural patient-specific questions regarding restrictions on the patient's abilities in daily activities, taking into account the patient's existing condition and medical history;
having an electronic expert system select from the database ones of the questions to be asked over a monitoring period of plural days and schedule when the selected questions are to be asked during the monitoring period;
electronically transmitting the selected questions at the scheduled times to the patient;
electronically receiving from the patient responses to the selected questions; and
having the expert system evaluate the patient's responses to the selected questions.

2. The method of claim 1, further comprising the step of having the expert system select further ones of the questions to ask and schedule when to ask the further selected questions based on the evaluation of the responses.

3. The method of claim 2, further comprising the steps wherein a predetermined response to one of the selected questions elicits asking a specific one of the further questions on a succeeding day, and rescheduling ones of the selected questions that had been scheduled to be asked on the succeeding day.

4. The method of claim 1, wherein the step of compiling the questions comprises the step of having the expert system compile the questions from a list of possible inquiries.

5. The method of claim 4, wherein the step of compiling the questions further comprises the step of further taking into account at least one of weather, pollen condition, time of year, and relevant epidemics when selecting the questions to be asked.

6. The method of claim 1, further comprising the step of receiving medical data from the patient electronically, and wherein the step of compiling the questions comprises the step of further taking into account the received medical data.

7. The method of claim 6, wherein the medical data includes the patient's blood pressure.

8. The method of claim 1, further comprising the step of having the expert system call for medical assistance for the patient based on the evaluation of the responses.

9. The method of claim 1, further comprising the step of compiling an electronic journal of the patient's responses.

10. The method of claim 1, further comprising the steps of compiling an electronically accessible list of further voluntary questions, making the list available to the patient electronically, and inviting the patient to respond electronically to ones of the further voluntary questions in the list.

11. A system for monitoring restrictions of a patient's abilities, the system comprising:
a database with plural patient-specific questions regarding restrictions on a patient's abilities in daily activities, the questions taking into account a patient's existing condition and medical history;
an electronic expert system connected to said database for selecting ones of the questions to be asked over a monitoring period of plural days and scheduling when the selected questions are to be asked during the monitoring period;
a communication apparatus connected to said expert system for electronically connecting said expert system and a patient and for transmitting the selected questions at the scheduled times to the patient and receiving from the patient responses to the selected questions; and
wherein said expert system evaluates a patient's responses to the selected questions.

12. The system of claim 11, wherein said expert system selects further ones of the questions to ask based on the evaluation of the responses and schedules when to ask the further selected questions.

13. The system of claim 12, wherein said expert system, upon receipt of a predetermined response to one of the selected questions, asks a specific one of the further questions on a succeeding day, and reschedules ones of the selected questions that had been scheduled to be asked on the succeeding day.

14. The system of claim 11, wherein said expert system compiles said database from a multiplicity of possible inquiries.

15. The system of claim 11, further comprising sensors for sensing patient medical data and communicating the sensed medical data to said expert system, and wherein said expert system selects the questions taking into account the received sensed medical data.

16. The system of claim 15, wherein said sensors include a blood pressure monitor.

17. The system of claim 11, further comprising a communication connection between said expert system and a source of medical assistance, and wherein said expert system automatically contacts the source of medical assistance based on the evaluation of a patient's responses.

18. The system of claim 11, wherein said expert system compiles an electronic journal of a patients responses.

19. The system of claim 11, wherein said database further comprises a list of further voluntary questions, and wherein said expert system makes said list available to a patient electronically, and invites a patient to respond electronically to ones of the further voluntary questions.
